(19) United States
(12) Patent Application Publication
(10) Pub. No.: US 2025/0179423 A1
(43) Pub. Date: Jun. 5, 2025

(54) POTENT HIGH YIELD FERMENTATION AND MANUFACTURING OF AKKERMANSIA MUCINIPHILA USING PLANT-BASED MUCIN AND ENCAPSULATION FOR STABILITY

(71) Applicant: Vidya Herbs, Inc., Fullerton, CA (US)

(72) Inventors: Kodimule Shyamprasad, Hassan (IN); Prabu Rajagopalan, Hassan (IN); Raksha Sunhare, Hassan (IN)

(21) Appl. No.: 18/953,300

(22) Filed: Nov. 20, 2024

Related U.S. Application Data
(60) Provisional application No. 63/604,264, filed on Nov. 30, 2023.

(51) Int. Cl. C12N 1/20; C12N 11/04; C12Q 1/06

(52) U.S. Cl. CPC C12N 1/20; C12N 11/04; C12Q 1/06

(57) ABSTRACT

Provided herein are systems and methods for Potent high yield fermentation and Manufacturing of Akkermansia muciniphila using plant-based Mucin and encapsulation for better stability.

### CROSS-REFERENCE TO RELATED APPLICATIONS

This application claims priority to U.S. provisional application Ser. No. 63/604,264, filed Nov. 30, 2023, herein incorporated by reference in its entirety.

### BACKGROUND

This invention relates to the development of thermochemical processes for high cell density post-biotic production.

Akkermansia muciniphila (A. muciniphila) is a gram-negative anaerobic bacterium, which is the first and only representative member of the Verrucomicrobia phylum found in the human intestinal tract. A. muciniphila is a mucus-colonizing member of the microbiota and may constitute up to 3% of the gut microbiota. This oval shape bacteria exhibits non-motile and non-pathogenic properties with strong metabolic activity. Based on the clinical significance it is known as a next generation beneficial strain for gut health. Its mucin degradation activity leads to the production of propionate and acetate. The abundance of A. muciniphila is significantly reduced in metabolic disorders, such as diabetes and obesity.

Akkermansia muciniphila is known for specific culture conditions and complex animal-based medium with mucin as sole carbon and nitrogen source. Akkermansia muciniphila mainly grown in mucin (Animal based) medium but also can be grow on a limited number of sugars including N-acetylglucosamine, N-acetylgalactosamine, and glucose or a synthetic medium constituted of glucose, N-acetylglucosamine, peptone, and threonine that has been confirmed to be safe for human administration. Akkermansia muciniphila was first isolated from a fecal sample in anaerobic medium containing gastric mucin (its sole energy source) A. muciniphila was discovered to directly bind to enterocytes to enable colonization, while its degradation of mucin was identified to stimulate mucin production and increase mucin thickness, thereby strengthening epithelial integrity.

High startup costs, complicated media, and sluggish response times are significant barriers to the development of new fermentation-based production systems in the bioeconomy. High cell density cultures are an efficient way to increase the volumetric productivity of fermentation processes, allowing for the use of smaller reactors and faster, more dependable procedures. Biochemical and process modification is a commercially viable alternative to genetic modification and feed batch methods to achieve high cell density.

The present invention attempts to solve these problems, as well as others.

### SUMMARY OF THE INVENTION

Provided herein are systems and methods for Potent high yield fermentation and Manufacturing of Akkermansia muciniphila using plant-based Mucin and encapsulation for better stability.

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53 (c) EPC and Rule 28 (b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

### BRIEF DESCRIPTION OF THE DRAWINGS

In the accompanying figures, like elements are identified by like reference numerals among the several preferred embodiments of the present invention.

FIG. 1 is a cross-sectional view of the stirred tank fermenter diagram, according to one embodiment.

FIG. 2 is a schematic flow chart for high cell density fermentation, according to one embodiment.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, biological, biochemical, and mechanical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### DESCRIPTION OF EMBODIMENTS

Generally speaking, a method of manufacturing using plant-based mucin related compounds rather than regular conventional animal-based mucin is disclosed herein and a commercial long-term feasibility has been achieved and ETP related process can be avoided. High startup costs, complicated media, and sluggish response times are significant barriers to the development of new fermentation-based production systems in the bioeconomy. High cell density cultures are an efficient way to increase the volumetric productivity of fermentation processes, allowing for the use of smaller reactors and faster, more dependable procedures. Biochemical and process modification is a commercially viable alternative to genetic modification and feed batch methods to achieve high cell density. The method of high cell density production comprises potent high yield fermentation and manufacturing of Akkermansia muciniphila using plant-based Mucin and encapsulation for better stability.

A method of producing high cell density probiotics, comprising: preparing a bacterial suspension by inoculating a single colony of Akkermansia muciniphila in Brain Heart Infusion (BHI) broth under anaerobic conditions and incubating the colony at a temperature between about 30° C. and about 45° C. purged with air concentration of 5% CO₂ for between about 15 hours and about 20 hours; transferring the culture to a BHI broth and incubating a seed culture; growing the seed culture on a CO₂ orbital a shaker at a temperature between about 30° C. and about 45° C. in a flask containing a seed medium; placing an inoculum from the seed culture in an anaerobic bioreactor with a fermentation medium; using pH, temperature, and agitation speed controls in the anaerobic bioreactor; and keeping the temperature and initial stirring speed at above temperature and 200-2000 FPM. The method further comprises controlling the foam with a silicone-based antifoam.

A method of high cell density production under vegetative conditions, comprises: Using a media formulation as disclosed in Table 1; using fermentation parameters for high cell density production including a pH between about 4.0 and about 8.0, a tip speed between about 200 FPM and about 2000 FPM, a temperature between about 30° C. and about 45° C., a CO₂ between about 1.0 LPM and about 10.0 LPM, a working volume between about 60% and about 70%, and a seed volume between about 2% and about 10%. The method further comprises extracting the cells for each strain from the fermentation broth using a centrifuge with a flow rate of between about 1200-1800× g and about 8000-11000× g and an incubation time between about 2 minutes and about 6 minutes. The method further comprises monitoring the OD₆₆ₘₘ of the flow through material and adjusting the feed flow rate to maintain the cell free supernatant equated to less than 5% of the cell concentration of the starting material; concentrating the resultant cell at the end of a single pass and re-suspending the concentrate into a stabilization buffer; washing the resuspension two more times. The method further comprises measuring the feed, supernatant, and cell concentrate fraction volumes, and using the associated cell concentrations to calculate the total cell for each of the three centrifuge passes. The method further comprises encapsulating the centrifuge supernatant by forming a continuous thin coating is formed around solid particles; resuspending the final centrifuge with a Tapioca starch with a ratio between about 1:1 to about 3:1 to wet cake to form a cream; spray drying the cream with an inlet temperature between about 100° C. and about 170° C., an outlet temperature between about 80° C. and about 110° C., and a relative humidity (RH) at about 25% to obtain a final spray dried powder. The method further comprises diluting the final spray dried power and resuspending the dilution in a buffer between about 20 and about 30 minutes in a CO₂ orbital shaker between about 20 rpm and 150 rpm. The method further comprises counting colonies and include Akkermansia muciniphila with a fermentation and complete sporulation time between about 18 hours and 20 hours, a packed cell volume (PCV) between about 1.6% and 3.1%, a Final OD between about 6.72 and about 8.9, a Dry biomass less than about 5.5 g/L and a 100 billion CFU/g.

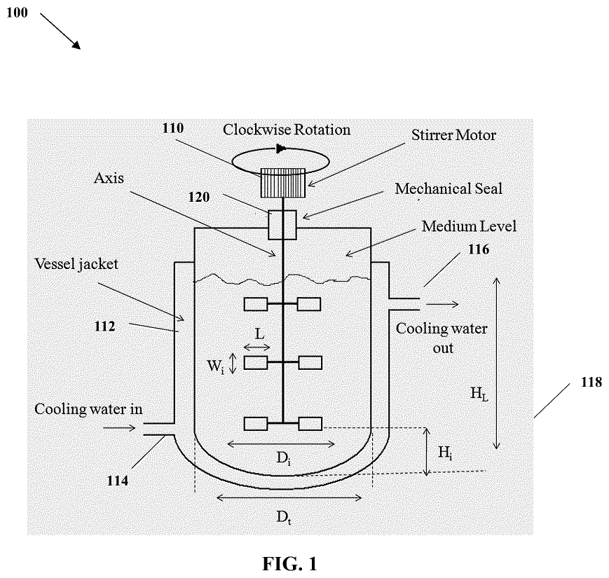

As shown in FIG. 1, an anaerobic bioreactor & shake flask are used as fermenters were fabricated to maintain the temperature inside the tank. The fermenter 100 includes a temperature, pH, and dissolved oxygen tension (DOT) control system. The fermenter 100 includes a stirrer 110 to stir about axis, a vessel jacket 112, cooling water intake 114, a cooling water outtake 116, and a mechanical seal 120. The fermenter 100 includes a HL and the stirrer 110 includes a plurality of stir elements 118 that include a length L and a Width Wi. The stirrer 110 includes a diameter Di. The bottom of the stirrer 110 includes a height Hi from the bottom of the fermenter 100. The fermenter 100 includes a diameter Dt and medium level of the fluid. During the fermentation, the foam was controlled manually with addition few drops of antifoam (Silicone based antifoam, Sigma) if required.

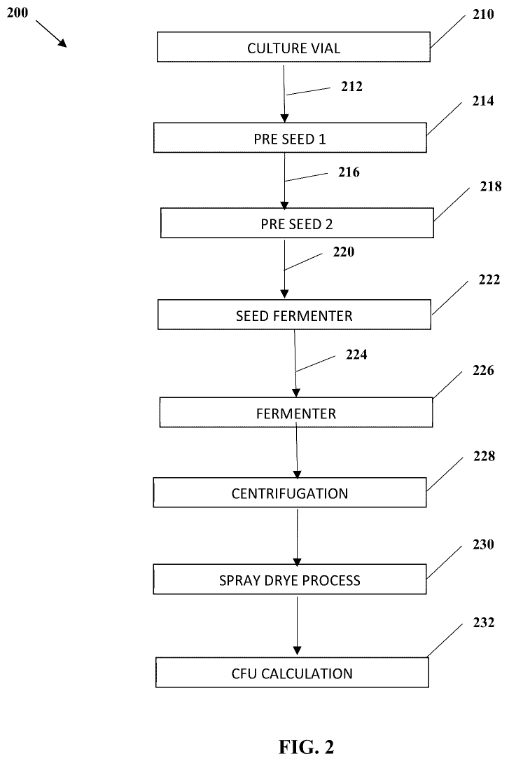

As shown in FIG. 2, the process flow for high cell density production 200 includes a culture vial step 210 where after freeze thawing, bring to Room Temperature and inoculate into seed media (Table 1) in process 212 to obtain pre-seed 1 at step 214. In process 216, after about 12 hours of incubation in shaker at 30-50 degrees C., the OD is checked along with the pH by microscopy to confirm for purity then it is transfer to seed media 2. In step 218 for the pre-seed 2, the process 220 then after about 8 hours of incubation in shaker at between 30-50 degrees C., the OD is checked microscopy and the pH to confirm for purity, then the pre-seed 2 is transferred to seed fermenter in step 222. In process 224, after about 8 hours in the seed fermenter, the OD is checked by microscopy and the pH to confirm for purity, then it is transferred to main fermenter in step 226. In step 228, the media is centrifuged, then undergoes a spray drying process in step 230, and then the CFU is calculated in step 230. The result is a spray-dried powder containing at least 100 billion CFU per gram. A plant-based mucin-related compound was used throughout the commercial production process and no Animal based Porcine or Mucin was used.

As compared to the regular conventional fermentation cycle which consists of mainly 36-40 hrs., this process and method has been reduced substantially to 20 hours, which brings an increased efficiency in terms of lower operating cost and commercial viability. Encapsulation was achieved by using Tapioca starch as an encapsulating agent and it provides protection against moisture, temperature and oxygen and the better stability of Akkermansia was achieved along with high yield. The recovery process is quite robust and high yield with higher potency (Colony forming unit) greater than 100 billion CFU per gram.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Scope of the Study

The current study focused solely on commercial Akkermansia muciniphila production using Plant-mucin related compounds in various synthetic medium. This process was commercialized with the lab to the 20 KL fermentation size, along with an encapsulation process to improve stability and high yield.

### Materials and Methods

### Bacterial Strains

This strain Akkermansia muciniphila is used for commercial production. The strains listed below were obtained and isolated from various sources: Akkermansia muciniphila VHAKM-76 and Akkermansia muciniphila BAA-835.

### Preparation of Bacterial Suspension

A single colony of Akkermansia muciniphila was inoculated in 10 mL Brain Heart Infusion (BHI) broth from a BHI plate under anaerobic conditions and incubated at 30-45° C. purged with 5% CO₂ in 10-150 rpm for 15-20 h. After incubation, 1 mL culture was transferred to fresh 100 mL BHI broth and incubated as described above.

A seed culture was grown on CO₂ orbital a shaker at 30-45° C. in 500 mL in screw cap Erlenmeyer flask (containing 150 mL of seed medium). A 200 mL inoculum was placed in a 5 L anaerobic bioreactor with 2 L of fermentation medium. The anaerobic bioreactor had pH, temperature, and agitation speed controls. The temperature and initial stirring speed were both kept at above temperature and 200-2000 FPM.

### Fermenter

The anaerobic bioreactor 20 KL, 5 KL 1000 L and 14 L & shake flask (500 mL) were used in this study. These fermenters were fabricated to maintain the temperature inside the tank. The schematic diagram, design, and parameters are depicting in FIG. 1. The fermenter was well equipped with temperature, pH, and dissolved oxygen tension (DOT) control system. During the fermentation, the foam was controlled manually with addition few drops of antifoam (Silicone based antifoam, Sigma) if required.

### High Cell Density Cultivation:

Indeed, high cell density cultivations are becoming increasingly important as several products develop, but also as a probiotic ingredient to add to their product, and thus their biomass is becoming increasingly valuable. The following media compositions were used for high cell density production under vegetative conditions. The media formulation is given table 1 and fermentation process in Table 2.

TABLE 1
Media composition for high cell density production of probiotics
* Hence VH XX are masked with full name of active ingredients.

| S No | Ingredients | g/L |
|---|---|---|
| 1 | VH-N 106 | 10.0-20.0 |
| 2 | VH-N 108 | 10.0-20.0 |
| 3 | VH-N 109 | 1.0-5.1 |
| 4 | VH-N 111 | 0.1-1.2 |
| 5 | VH-N-023 | 1.0-3.0 |
| 6 | VH-N-144 | 1.0-3.0 |
| 7 | VH-P 201 | 0.1-0.8 |
| 8 | VH-P 202 | 5.0-10 |
| 9 | VH-P 204 | 5.0-12 |
| 10 | VH-P 206 | 0.3-1.0 |

TABLE 2
fermentation parameters for high cell density production

| S No | Fermentation parameters | Ranges |
|---|---|---|
| 1 | pH | 4.0-8.0 |
| 2 | Tip speed (FPM) | 200-2000 |
| 3 | Temperature (° C.) | 30-45 |
| 4 | CO₂(LPM) | 1.0-10.0 |
| 5 | Working volume (%) | 60-70 |
| 6 | Seed volume (%) | 2-10 |

FIG. 2 is a process flow for high cell density production.

### Down Stream Process and Encapsualtion

The above cells for each strain were extracted from the fermentation broth using a centrifuge (Clara 200) with a flow rate of 1200-1800, 8000-11000× g and an incubation time of 2-6 minutes.

The OD₆₆₀ₙₘ of the flow through material was monitored and the feed flow rate appropriately adjusted to maintain the cell free supernatant which equated to less than 5% of the cell concentration of the starting material. The resultant cell concentrated at the end of a single pass was re-suspended into stabilization buffer and then washed two more times using the same method. The feed, supernatant, and cell concentrate fraction volumes were measured, and the associated cell concentrations were used to figure out total cell for each of the three centrifuge passes.

Then the final process involves the encapsulation of the centrifuge. Encapsulation can be defined as a process where a continuous thin coating is formed around solid particles. The final centrifuge was resuspended with Tapioca starch with 1:1 to 3:1 to wet cake. This cream was spray dried (with inlet temperature 100-170° C., outlet temperature 80-110° C., and Relative humidity (RH) 25%) to obtain the final spray dried powder. In the past, a wide range of products which were technically not feasible for manufacture but are possible today through the development and design of encapsulated ingredients. Such formulations derive from processes that totally envelop the active material in a coating or “encapsulated matric,” thereby conferring distinct physicochemical capabilities compared to the original non-encapsulated ingredients and acts as a barrier and provides protection against moisture, temperature and oxygen exposure and thus enabling the final Akkermansia strain with high potency and stable ingredient for wider form of application.

### Analysis Method for Probiotic Spray Dried Strain

The spray dryer powder described above was diluted and resuspended in saline buffer or phosphate buffer (pH 7, 0.1-0.3 M) for 20-30 minutes in a CO₂ orbital shaker with 20-150 rpm.

The microscopic examination to be taken of this resuspended powder suspension (every 10 minutes) looks for single colonies that may spew out due to its clumping nature. This process must be repeated until all single colonies appear with no clumping in the suspension. Following that, the samples were given the proper dilutions for the pour and spread plate method using BHI plates and incubated at 35-45° C. for 48-72 hours. The colony was counted, and complete calculation was performed using dilution factors.

TABLE 3
RESULTS

| | Fermentation | | | | |
|---|---|---|---|---|---|
| | and complete | | | Dry | |
| | sporulation | PCV | Final | biomass | |
| Culture name | time (hrs) | (%) | OD | (g/L) | CFU/g |
| Akkermansia | 18 ± 2 | 1.8-3.1 | 8.9 ± 2 | >5.5 | NLT 100 |
| Muciniphila | | | | | Billion |
| VHAKM-76 | | | | | |
| Akkermansia | 20 ± 2 | 1.6-2.7 | 6.75 ± 2 | >4.9 | NLT100 |
| Muciniphila | | | | | Billion |
| BAA-835 | | | | | |

Advantage of this process include obtaining a spray-dried powder containing at least 100 billion CFU per gram, the color of spray dried powder: Off white to light straw color & not in brown or soil color, a fine & free flow powder and not sticky in nature, animal-based mucin can be avoidable for commercial production process. As compared to regular conventional fermentation cycle which consists of mainly 36-40 hrs., this process has been reduced to 20 hours which brings an increased efficiency in terms of lower operating cost and commercial viability. Encapsulation was achieved by using Tapioca starch as an encapsulating agent and it provides protection against moisture, temperature and oxygen and the better stability of Akkermansia was achieved along with high yield.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A method producing high cell density probiotics, comprising:
a. Preparing a bacterial suspension by inoculating a single colony of Akkermansia muciniphila in Brain Heart Infusion (BHI) broth under anaerobic conditions and incubating the colony at a temperature between about 30° C. and about 45° C. purged with air concentration of 5% CO₂ for between about 15 hours and about 20 hours;
b. Transferring the culture to a BHI broth and incubating a seed culture;
c. Growing the seed culture on a CO₂ orbital a shaker at a temperature between about 30° C. and about 45° C. in a flask containing a seed medium;
d. Placing an inoculum from the seed culture in an anaerobic bioreactor with a fermentation medium; using pH, temperature, and agitation speed controls in the anaerobic bioreactor;
e. Keeping the temperature and initial stirring speed at above temperature and 200-2000 FPM.

2. The method of claim 1, further comprising controlling the foam with a silicone-based antifoam.

3. A method of high cell density production under vegetative conditions, comprising:
a. Using a media formulation as disclosed in Table 1;
b. Using fermentation parameters for high cell density production including a pH between about 4.0 and about 8.0, a tip speed between about 200 FPM and about 2000 FPM, a temperature between about 30° C. and about 45° C., a CO₂ between about 1.0 LPM and about 10.0 LPM, a working volume between about 60% and about 70%, and a seed volume between about 2% and about 10%.

4. A method of claim 3, further comprising extracting the cells for each strain from the fermentation broth using a centrifuge with a flow rate of between about 1200-1800× g and about 8000-11000× g and an incubation time between about 2 minutes and about 6 minutes.

5. The method of claim 4, further comprising monitoring the OD₆₆₀ₙₘ of the flow through material and adjusting the feed flow rate to maintain the cell free supernatant equated to less than 5% of the cell concentration of the starting material; concentrating the resultant cell at the end of a single pass and re-suspending the concentrate into a stabilization buffer; washing the resuspension two more times.

6. The method of claim 5, further comprising measuring the feed, supernatant, and cell concentrate fraction volumes, and using the associated cell concentrations to calculate the total cell for each of the three centrifuge passes.

7. The method of claim 6, further comprising encapsulating the centrifuge supernatant by forming a continuous thin coating is formed around solid particles; resuspending the final centrifuge with a Tapioca starch with a ratio between about 1:1 to about 3:1 to wet cake to form a cream; spray drying the cream with an inlet temperature between about 100° C. and about 170° C., an outlet temperature between about 80° C. and about 110° C., and a relative humidity (RH) at about 25% to obtain a final spray dried powder.

8. The method of claim 7, further comprising diluting the final spray dried power and resuspending the dilution in a buffer between about 20 and about 30 minutes in a CO₂ orbital shaker between about 20 rpm and 150 rpm.

9. The method of claim 8, wherein the colonies are counted and include Akkermansia muciniphila with a fermentation and complete sporulation time between about 18 hours and 20 hours, a PCT between about 1.6% and 3.1%, a Final OD between about 6.72 and about 8.9, a Dry biomass less than about 5.5 g/L and a 100 billion CFU/g.
